(19) United States
(12) Patent Application Publication
(10) Pub. No.: US 2018/0178495 A1
(43) Pub. Date: Jun. 28, 2018

(54) Hydrophilic Coating Methods for Chemically Inert Substrates

(71) Applicant: Xiaoxi Kevin Chen, Natick, MA (US)

(72) Inventor: Xiaoxi Kevin Chen, Natick, MA (US)

(21) Appl. No.: 15/857,527

(22) Filed: Dec. 28, 2017

Related U.S. Application Data
(60) Provisional application No. 62/439,869, filed on Dec. 28, 2016.

(51) Int. Cl. B32B 27/08; B32B 27/32; B32B 15/08; H01J 37/32; C08L 27/12; C08L 83/04; C08J 3/28; C08J 3/24

(52) U.S. Cl. CPC B32B 27/08; B32B 27/322; B32B 15/08; H01J 37/32018; C07C 31/02; C08L 83/04; C08J 3/28; C08J 3/24; C08L 27/12

(57) ABSTRACT

The present invention discloses methods for producing a hydrophilic coating for chemically inert substrates such as fluoropolymers using a multi-step coating process consisting of (1) producing a plasma polymerization coating of alcohol compounds on the substrate, followed by (2) sequentially contacting the plasma polymer coated substrate with one or more solutions of hydrophilic polymers. Advantageously, such methods produce a strongly adhered hydrophilic coating for fluoropolymer and other chemically inert substrates.

### CROSS-REFERENCE TO RELATED APPLICATION

This application claims priority of U.S. Provisional Patent Application No. 62/439869, filed Dec. 28, 2016, the entire contents of which are incorporated by reference herein.

### FIELD OF THE INVENTION

The present invention discloses methods for producing a hydrophilic coating for chemically inert substrates such as fluoropolymers using a multi-step coating process consisting of (1) producing a plasma polymerization coating of alcohol compounds on the substrate, followed by (2) sequentially contacting the plasma polymer coated substrate with one or more solutions of hydrophilic polymers. Advantageously, such methods produce a strongly adhered hydrophilic coating for fluoropolymers and other chemically inert substrates.

### BACKGROUND OF THE INVENTION

Chemically inert materials have been widely used in medical device applications, especially for implanted devices, catheters, guidewires, and graft material in surgical interventions. Examples of chemically inert materials used in medical applications include fluoropolymers, polyether ether ketone (PEEK), silicone elastomers, Nylon, polyether block amides (PEBAX), etc. Examples of fluoropolymers used in medical applications include polytetrafluoroethylene (PTFE), polyethyl enetetrafluoroethylene (ETFE), fluorinated ethylene-propylene (FEP), and perfluoroalkoxy polymer (PFA). Most of these chemically inert materials are hydrophobic: they cannot be wet by water or water containing substance.

There are applications in which it is advantageous to coat chemically inert substrates with a hydrophilic coating. For example, in the medical guidewire applications, it is advantageous to use a hydrophilic coating to improve the lubricity of guidewires; in medical catheter applications, it is advantageous to use a hydrophilic coating to reduce bacterial adhesion and/or reduce thrombus formation.

Coating of chemically inert substrates with hydrophilic coatings has been a significant challenge due to the following: (1) The chemical inertness makes it difficult to create chemically reactive groups on the surface to crosslink with the hydrophilic polymer; (2) The hydrophobicity of the surface makes it difficult for the coating solution, which usually contains water, to remain on the surface; (3) The non-stick property of the surface makes it difficult for a hydrophilic polymer to adhere well on the substrate surface.

Prior arts of applying a hydrophilic coating include dip coating, spray coating, dip/spray coating followed by UV curing or thermal curing. These methods have yield poor results on chemically inert substrates.

### SUMMARY OF THE INVENTION

A method is disclosed herein for applying a hydrophilic coating on chemically inert substrates by first coating the substrates with plasma polymerization of alcohol compounds, followed by coating the substrates with one or more solutions of hydrophilic polymers.

In the first step of coating, the substrates are exposed to plasma glow discharge in the presence of vapors of one or more alcohol compounds. The plasma polymer of the alcohol compounds is coated on the substrates.

In the following steps of coating, the substrates are brought into contact with one or more solutions of hydrophilic polymers. This may consist of a sequential dipping/soaking of the substrates in different solutions, allowing one or more layers of hydrophilic polymers to coat on the substrates.

One advantage of the disclosed method is that the plasma polymer of alcohol compounds adheres strongly on chemically inert substrates, permanently changing the property of the surface.

A further advantage of the disclosed method is that the hydrophilic polymers adhere strongly on the plasma polymer of alcohol compounds. This method overcome the challenges for coating chemically inert substrates directly.

These and other features of the invention will be better understood through a study of the following detailed description and accompanying drawings.

### BRIEF DESCRIPTION OF THE FIGURES

FIG. 1 is a drawing representing a chemically inert substrate coated using subject invention multi-step coating method comprising of a plasma polymerization coating step and a hydrophilic polymer coating step.

### DETAILED DESCRIPTION OF THE INVENTION

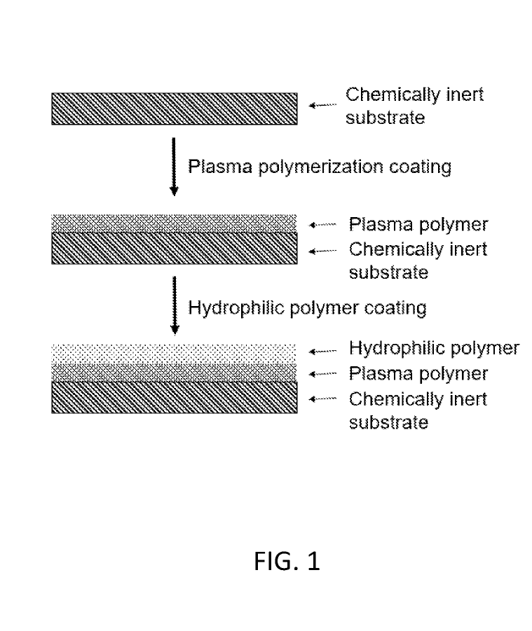

With reference to FIG. 1, a chemically inert substrate is first coated with plasma polymerization of alcohol compounds, and then the plasma polymer coated substrate is coated with a layer of hydrophilic polymer.

Any known technique can be used to generate the plasma glow discharge for plasma polymerization coating. The plasma may be generated using AC or DC power, radio-frequency (RF) power or micro-wave frequency power. Preferably, the plasma system is driven by a single radio-frequency (RF) power supply; typically at 13.56 MHz. The plasma system can either be capacitively coupled plasma, or inductively coupled plasma.

In a preferred embodiment, the monomer used for plasma polymerization is selected from methanol, ethanol, isopropanol, butanol, and pentanol. In the plasma state, the alcohol compounds are ionized and react with the surface of the substrate, forming a covalently bound thin film containing hydroxyl groups.

Any known technique can be used to produce the hydrophilic coating on top of the plasma polymer coating. The coating may be performed using dip coating, soak coating or spray coating. One or more solutions can be used to produce the hydrophilic coating with one or more layers of hydrophilic polymer. Each solutions may contain a mixture of polymers. After the application of each solution, the substrates may be rinsed with water or an organic solvent.

### EXAMPLES

### Example 1

PTFE substrates (squares, 1 inch by 1 inch) were coated in a radiofrequency plasma glow discharge chamber in the presence of vapors of isopropanol. The chamber was first pumped down to a pressure below 0.04 Torr. The vapor of isopropanol was introduced into the chamber and the pressure is adjusted to 0.2 Torr. A radiofrequency power of approximately 100 W was applied for 20 minutes to generate the plasma polymer coating.

### Example 2

PTFE substrates coated with plasma polymer in Example 1 was dipped in a solution of 5% (w/v) polyvinylpyrrolidone (PVP) in 90% ethanol for 5 seconds, then dried at room temperature in air. As a control, PTFE substrates which have not been coated with plasma polymer were coated with PVP in the same way. The PTFE substrates coated with plasma polymer followed by PVP was found to be hydrophilic and lubricious. The PTFE substrates coated with PVP directly without plasma polymer was found to have no coating adhering to the substrate. The coated substrates were rinsed with water extensively, dried and characterized by Sessile drop contact angle measurements. The results are summarized in the Table below:

| | Water Contact |
|---|---|
| PTFE substrates | Angle |
| Control 1: Uncoated | 110° ± 5° |
| Control 2: attempted coating with PVP | 110° ± 5° |
| without plasma polymer (Example 2 control) | |
| Current invention: coated with plasma | 35° ± 5° |
| polymer, then PVP (Example 2) | |

### Example 3

PTFE substrates coated with plasma polymerization in Example 1 was soaked in a solution of 1% (w/v) polyethylenimine (PEI) for 1 hour, rinsed with water, then soaked in a solution of 1% (w/v) oxidized dextran for 1 hour, and finally rinsed with water. As a control, PTFE substrates which have not been coated with plasma polymerization were coated with PEI and dextran in the same way. The coated substrates were rinsed with water extensively, dried and characterized by Sessile drop contact angle measurements. The results are summarized in the Table below:

| | Water Contact |
|---|---|
| PTFE substrates | Angle |
| Control 1: Uncoated | 110° ± 5° |
| Control 2: Attempted coating with PEI/dextran | 65° ± 5° |
| without plasma polymer (Example 3 control) | |
| Current invention: coated with plasma | <20° |
| polymer, then PEI and dextran (Example 3) | |

### Example 4

PEEK substrates (squares, 1 inch by 1 inch) were coated in a radiofrequency plasma glow discharge chamber in the presence of vapors of isopropanol. The chamber was first pumped down to a pressure below 0.04 Torr. The vapor of isopropanol was introduced into the chamber and the pressure is adjusted to 0.2 Torr. A radiofrequency power of approximately 100 W was applied for 20 minutes to generate the plasma polymer coating.

### Example 5

PEEK substrates coated with plasma polymer in Example 4 was dipped in a solution of 5% (w/v) polyvinylpyrrolidone (PVP) in 90% ethanol for 5 seconds, then dried at room temperature in air. As a control, PEEK substrates which have not been coated with plasma polymer were coated with PVP in the same way. The PEEK substrates coated with plasma polymer followed by PVP was found to be hydrophilic and lubricious. The PEEK substrates coated with PVP directly without plasma polymer was found to have no coating adhering to the substrate.

### Example 6

Silicone elastomer substrates (squares, 1 inch by 1 inch) were coated in a radiofrequency plasma glow discharge chamber in the presence of vapors of isopropanol. The chamber was first pumped down to a pressure below 0.04 Torr. The vapor of isopropanol was introduced into the chamber and the pressure is adjusted to 0.2 Torr. A radiofrequency power of approximately 100 W was applied for 20 minutes to generate the plasma polymer coating.

### Example 7

Silicone elastomer substrates coated with plasma polymerization in Example 6 was soaked in a solution of 1% (w/v) polyethylenimine (PEI) for 1 hour, rinsed with water, then soaked in a solution of 1% (w/v) oxidized dextran for 1 hour, and finally rinsed with water. As a control, silicone elastomer substrates which have not been coated with plasma polymerization were coated with PEI and dextran in the same way. The coated substrates were rinsed with water extensively, dried and characterized by Sessile drop contact angle measurements. The results are summarized in the Table below:

| | Water Contact |
|---|---|
| Silicone elastomer substrates | Angle |
| Control 1: Uncoated | 110° ± 5° |
| Control 2: Attempted coating with PEI/dextran | 70° ± 5° |
| without plasma polymer (Example 7 control) | |
| Current invention: coated with plasma | <20° |
| polymer, then PEI and dextran (Example 7) | |

### Example 8

Nylon substrates (squares, 1 inch by 1 inch) were coated in a radiofrequency plasma glow discharge chamber in the presence of vapors of isopropanol. The chamber was first pumped down to a pressure below 0.04 Torr. The vapor of isopropanol was introduced into the chamber and the pressure is adjusted to 0.2 Torr. A radiofrequency power of approximately 100 W was applied for 20 minutes to generate the plasma polymer coating.

### Example 9

Nylon substrates coated with plasma polymerization in Example 8 was soaked in a solution of 1% (w/v) polyethylenimine (PEI) for 1 hour, rinsed with water, then soaked in a solution of 1% (w/v) oxidized dextran for 1 hour, and finally rinsed with water. As a control, Nylon substrates which have not been coated with plasma polymerization were coated with PEI and dextran in the same way. The coated substrates were rinsed with water extensively, dried and characterized by Sessile drop contact angle measurements. The results are summarized in the Table below:

| | Water Contact |
|---|---|
| Nylon substrates | Angle |
| Control 1: Uncoated | 70° ± 5° |
| Control 2: Attempted coating with PEI/dextran | 40° ± 5° |
| without plasma polymer (Example 9 control) | |
| Current invention: coated with plasma | <20° |
| polymer, then PEI and dextran (Example 9) | |

### Example 10

PEBAX substrates (squares, 1 inch by 1 inch) were coated in a radiofrequency plasma glow discharge chamber in the presence of vapors of isopropanol. The chamber was first pumped down to a pressure below 0.04 Torr. The vapor of isopropanol was introduced into the chamber and the pressure is adjusted to 0.2 Torr. A radiofrequency power of approximately 100 W was applied for 20 minutes to generate the plasma polymer coating.

### Example 11

PEBAX substrates coated with plasma polymer in Example 10 was dipped in a solution of 5% (w/v) polyvinylpyrrolidone (PVP) in 90% ethanol for 5 seconds, then dried at room temperature in air. As a control, PEBAX substrates which have not been coated with plasma polymer were coated with PVP in the same way. The PEBAX substrates coated with plasma polymer followed by PVP was found to be hydrophilic and lubricious. The PEBAX substrates coated with PVP directly without plasma polymer was found to have no coating adhering to the substrate.

### Example 12

Stainless steel substrates (squares, 1 inch by 1 inch) were coated in a radiofrequency plasma glow discharge chamber in the presence of vapors of isopropanol. The chamber was first pumped down to a pressure below 0.04 Torr. The vapor of isopropanol was introduced into the chamber and the pressure is adjusted to 0.2 Torr. A radiofrequency power of approximately 100 W was applied for 20 minutes to generate the plasma polymer coating.

### Example 13

Stainless steel substrates coated with plasma polymer in Example 12 was dipped in a solution of 5% (w/v) polyvinylpyrrolidone (PVP) in 90% ethanol for 5 seconds, then dried at room temperature in air. As a control, stainless steel substrates which have not been coated with plasma polymer were coated with PVP in the same way. The stainless steel substrates coated with plasma polymer followed by PVP was found to be hydrophilic and lubricious. The stainless steel substrates coated with PVP directly without plasma polymer was found to have no coating adhering to the substrate.

As will be appreciated by those skilled in the art, the subject invention can be used to produce a hydrophilic coating on chemically inert substrates. By way of non-limiting example, the subject invention can be used to coat medical devices such as catheter or guidewires that is made of or contains chemically inert polymers to increase hydrophilicity and lubricity.

What is claimed is:

1. A method for producing a hydrophilic coating for a chemically inert substrate using a multi-step process consisting of (a) exposing said substrate to a plasma glow discharge in the presence of the vapor of at least one alcohol compound to produce a plasma polymer coated substrate; (b) contacting said plasma polymer coated substrate with a solution of hydrophilic polymers to produce a hydrophilic polymer coated substrate.

2. A method of claim 1, wherein said substrate contains fluoropolymers.

3. A method of claim 1, wherein said substrate contains fluoropolymers selected from the following list: polytetrafluoroethylene (PTFE), polyethylenetetrafluoroethylene (ETFE), fluorinated ethylene-propylene (FEP), perfluoroalkoxy polymer (PFA), polyethylenechlorotrifluoroethylene (ECTFE), polychlorotrifluoroethylene (PCTFE), polyvinylidene fluoride (PVDF), polyvinylfluoride (PVF), perfluorinated elastomer, fluoroelastomer, chlorotrifluoroethylenevinylidene fluoride, perfluoropolyether, perfluorosulfonic acid, and perfluoropolyoxetane.

4. A method of claim 1, wherein said substrate contains polyether ether ketone (PEEK).

5. A method of claim 1, wherein said substrate contains silicone elastomers.

6. A method of claim 1, wherein said substrate contains Nylon.

7. A method of claim 1, wherein said substrate contains polyether block amides (PEBAX).

8. A method of claim 1, wherein said substrate contains metals.

9. A method of claim 1, wherein said multi-step process further consists of sequentially contacting said hydrophilic polymer coated substrate with one or more solvents or solutions of hydrophilic polymers to produce a multi-layer hydrophilic polymer coated substrate.

10. A method of claim 9, wherein said multi-layer hydrophilic polymer contain covalent linkage between layers of hydrophilic polymers.

11. A method of claim 1, wherein said alcohol compound is selected from methanol, ethanol, isopropanol, butanol, and pentanol.
